(19) United States
(12) Patent Application Publication
(10) Pub. No.: US 2008/0003681 A1
(43) Pub. Date: Jan. 3, 2008

(54) Methods for altering cellular susceptibility to infection

(76) Inventors: Mahalaxmi Gita Bangera, Renton, WA (US); Leroy E. Hood, Seattle, WA (US); Roderick A. Hyde, Livermore, CA (US); Muriel Y. Ishikawa, Livermore, CA (US); Edward K.Y. Jung, Bellevue, WA (US); Robert Langer, Newton, MA (US); Elizabeth A. Sweeney, Seattle, WA (US); Lowell L. Wood, Livermore, CA (US); Victoria Y.H. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(21) Appl. No.: 11/478,316

(22) Filed: Jun. 28, 2006

(51) Int. Cl. C12N 15/00; C12N 5/06

(52) U.S. Cl. 435/455; 435/325

(57) ABSTRACT

Methods as described herein relate to altering the potential infectivity of a cell or cells. Included are methods for altering DNA of non-terminally differentiated cells in locations that inhibit the function of at least one CCR5 gene relative to an unaltered version of the gene and providing stimulus for differentiation of the altered cell.

### SUMMARY

In one aspect, a method includes but is not limited to altering DNA of a non-terminally differentiated cell in a location that inhibits function of at least one CCR5 gene relative to an unaltered version of the gene and providing stimulus for differentiation of the altered cell. In addition to the foregoing, other method and cell line aspects are described in the claims, drawings, and text forming a part of the present application.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 shows a flow diagram of steps included in some embodiments of the methods described herein.

FIG. 2 shows a flow diagram of steps included in some embodiments of the methods described herein.

FIG. 3 shows a flow diagram of steps included in further embodiments of the methods described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

Methods described herein include altering DNA of a non-terminally differentiated cell in a location that inhibits function of at least one CCR5 gene relative to an unaltered version of the gene and providing stimulus for differentiation of the altered cell. Methods also include maintaining a cell ex-vivo in a non-terminally differentiated state, altering DNA of the cell in a manner that inhibits function of at least one CCR5 gene relative to an unaltered version of the gene and providing stimulus for differentiation of the altered cell. Further methods include creating a genomic deletion in at least one CCR5 gene in a non-terminally differentiated cell and regulating differentiation of the cell. Also described herein are ex-vivo cell lines comprising a plurality of cells containing DNA structure that includes an induced alteration in at least one CCR % gene and at least one cell in a differentiated state.

It is envisioned that the methods and cell lines described herein will be useful for research into the infectivity and disease progression of pathogens that utilize CCR5 to infect and invade cells. Moreover, since methods and cell lines as described herein also provide for the differentiation of cells with inhibited CCR5 function, cell types of varying stages of differentiation with similar inhibited function of their CCR5 genes may be examined for research purposes. In addition it is envisioned that the methods and cell lines disclosed herein will be applicable for treatments of individuals infected with or exposed to pathogens that rely on CCR5 to infect and invade cells. It is also possible that methods and cell lines disclosed herein would be applicable for use on a widespread scale throughout a population to reduce or mitigate infection.

The DNA alteration(s) may be made by any one of a number of techniques known to those of skill in the art and/or as disclosed herein. For example, one group of methods used for altering DNA is mutagenesis by chemical means such as nitrosoguanidine, aflatoxin B1 (AFB1), DNA intercalculating agents such as ethidium bromide, base analogs such as 5-bromo-2-deoxyuridine (BrdU), alkalating agents such as N-ethyl-N-nitrosourea (ENU), methylating agents such as ethane methyl sulfonate (EMS), and DNA crosslinkers such as platinum. Also by way of example, there are a group of methods for altering DNA by mutagenesis by radiological means including the use of ultraviolet (UV) and ionizing radiation. As further example, another type of method for the alteration of DNA includes the use of zinc-finger proteins and double strand breaks to introduce specific alterations in a targeted manner, as described in Urnov et al., “Highly Efficient Endogenous Human Gene Correction Using Designed Zinc-finger Nucleases” Nature 435: 646-651 (2005), which is herein incorporated by reference. Methods as described herein may also use alteration creation methods that make use of chimeric oligonucleotides to introduce specific alterations, as described in Liu et al., “In Vivo Gene Repair of Point and Frameshift Mutations Directed by Chimeric RNA/DNA Oligonucleotides and Modified Single-Stranded Oligonucleotides”, Nucleic Acids Research 29(20): 4238-4250 (2001) and Graham and Dickson, “Gene Repair and Mutagenesis Mediated by Chimeric RNA-DNA Oligonucleotides: Chimeraplasty for Gene Therapy and Conversion of Single Nucleotide Polymorphisms (SNPs)”, Biochimica et Biophysica Acta 1587:1-6 (2002), which are herein incorporated by reference. Alterations may also be introduced through the use of many techniques that are based on exogenous nucleotides to alter endogenous gene expression. Some of these techniques are described in Patil et al., “DNA-based Therapeutics and DNA Delivery Systems: A Comprehensive Review”, AAPS Journal 7(1): Article 9 (2005) as well as in Juliano et al., “Epigenetic Manipulation of Gene Expression: a Toolkit for Cell Biologists”, Journal of Cell Biology 169(6): 847-857 (2005), which are herein incorporated by reference. There may be at least one alteration introduced by any method and may be of any type, including an insertion, a deletion, a substitution, an inversion, a change in methylation state or substantially any combination of these. The DNA alteration may be altering genomic DNA. The DNA that is altered can be chromosomal or non-chromosomal and can be altered in either a permanent or transient manner. In some embodiments, a single alteration will be made while in others there will be several or a series of alterations. Further information regarding DNA alterations may be found in chapter 16 of Principles of Genetics (second edition); James W. Fristrom and Michael T. Clegg, Chiron Press 1988.

Depending on the embodiment, alterations may be made in either coding or non-coding regions of DNA. The “coding” regions of a gene are the portions of the gene which are translated into RNA and may also be transcribed into protein. The “non-coding” regions of a gene are the DNA regions of a gene and the surrounding chromosome that are not directly transcribed into RNA, but their DNA sequence and/or structure influences the transcription of the coding region. The non-coding region of a gene includes regulatory sequences which influence the transcription of the coding regions, but is not limited to those sequences. Examples of regulatory sequences include transcription factor binding sites, RNA splice sites, initiation sites as well as RNA and protein processing and localization factor recognition sites. In some situations, the non-coding region of a gene may extend to regions of DNA that are not directly adjacent to the coding region. A further discussion regarding coding and non-coding regions of genes may be found in Chapter 2 of Genes VII; Benjamin Lewin, Oxford University Press 2000, which is herein incorporated by reference.

In some embodiments, DNA is altered in a location that inhibits function of at least one CCR5 (chemokine (C-C motif) receptor 5) gene, which in humans (Homo sapiens) is also referred to as CKR5, CD195, CKR-5, CCCKR5, CMKBR5 and CC-CKR-5 (GenBank Accession Number NM_{—}000579 and GeneID 1234 as of June 2006). The CCR5 protein is known to be a cell surface receptor. Several CCR5 gene homologs from diverse species are part of the UniGene Cluster with reference number Hs. 450802 (as of June 2006). The CCR5 gene is located on chromosome 3p21 in the human. A description of the genomic organization of the human CCR5 gene, including information regarding coding and non-coding regions, may be found in Mummidi et al., “The Human CC Chemokine Receptor 5 (CCR5) Gene”, Journal of Biological Chemistry, 272(49): 30662-30671 (1997), which is herein incorporated by reference. The protein sequence corresponding to the human CCR5 gene may be obtained from the NCBI Entrez Protein database by reference to accession number P51681 (as of June 2006). A number of alterations in the CCR5 gene have been described in both the coding and non-coding regions of the gene. These include alterations in regulatory regions such as a CCR5-59353 T to C alteration, a CCR5-59356 C to T alteration and a CCR5-59402 A to G alteration (see Kostrikis et al., “A Polymorphism in the Regulatory Region of the CC-Chemokine Receptor 5 Gene Influences Perinatal Transmission of Human Immunodeficiency Virus Type 1 to African-American Infants”, Journal of Virology 73(12): 10264-10271 (1999), which is herein incorporated by reference). Various alterations within the coding region have also been described that are insertion, deletion, substitution and inversion alterations. A more detailed listing of both coding and non-coding alterations within the CCR5 gene may be found in the Online Mendelian Inheritance in Man (OMIM) listing for the gene (reference number 601373 as of June 2006). In some embodiments of the methods described herein, the altering DNA creates a DNA sequence within at least one CCR5 gene with a 32 nucleotide deletion encompassing nucleotides 794 through 825 of GenBank accession number NM_{—}000579 (as of June 2006). For more information regarding alterations within the CCR5 gene, see also Dean et al., “Genetic Restriction of HIV-1 Infection and Progression to AIDS by a Deletion Allele of the CKR5 Structural Gene”, Science 273:1856-1862 (1996), Samson et al., “Resistance to HIV-1 Infection in Caucasian Individuals Bearing Mutant Alleles of the CCR-5 Chemokine Receptor Gene”, Nature 382:722-725 (1996) and Liu et al., “Homozygous Defect in HIV-1 Coreceptor Accounts for Resistance of Some Multiply-Exposed Individuals to HIV-1 Infection”, Cell 86:367-377 (1996) which are herein incorporated by reference. In some embodiments, the altering DNA creates a DNA sequence within at least one CCR5 gene that encodes a protein sequence with a frameshift after codon 174 and a truncation at codon 206 relative to the unaltered protein sequence relative to the CCR5 protein sequence, which is available in the NCBI Entrez Protein database with accession number P51681.

In some embodiments, non-human systems are involved and the corresponding non-human homolog of the CCR5 gene is inhibited. Sequences of several homologs of the CCR5 gene from diverse species may be obtained through the UniGene Cluster for the gene, which has accession number Hs. 450802 (as of June 2006). For example, in the mouse (Mus musculus) the CCR5 homolog is generally known as Ccr5, although it has also been known as AM4-7, CD195 and Cmkbr5. Ccr5 is located on murine chromosome 9 and has Genbank GeneID 12774 (as of June 2006). As a further example, in rat (Rattus norvegicus) the CCR5 homolog is generally known as Ccr5, although it has also been known as Ckr5 and Cmkbr5, and is located on chromosome 8 with the GenBank GeneID 117029 (as of June 2006). As a further example, in the domestic dog (Canis familiaris) the CCR5 homolog is generally known as CCR5, although it has also been known as GPCR, and is located on canine chromosome 20 with the GenBank GeneID 484789 (as of June 2006). As further example, in the domestic pig (Sus scrofa) the CCR5 homolog is known as CCR5 and is located on porcine chromosome 13 with the GenBank GeneID 414371 (as of June 2006).

As is known to those of skill in the art, alterations can be introduced into DNA in location(s) that inhibit the function of a gene relative to an unaltered version of the gene. The alteration may occur ex-vivo. In some embodiments, the introduced alteration will be identifiable as introduced using standard molecular genetics techniques. For example, if the alteration results in the addition of additional DNA as a portion of a cloning procedure, that additional DNA will be apparent in DNA sequence analysis. Similarly, an alteration that is introduced via an extrachromosomal mechanism would be apparent at a chromosomal level. In some embodiments, alteration(s) will be introduced within the coding region of a gene itself, but they may also be introduced into non-coding region(s), which may be portions of the regulatory region(s). In some embodiments, the location(s) where the alteration(s) are introduced will be in cis to the gene that has its function inhibited, while in other embodiments it will be in trans or extra-chromosomal to the gene that has its function inhibited. Further discussion regarding control of gene expression may be found in Chapter 10 of The Molecular Biology of the Cell (second edition); Bruce Alberts, Dennis Bray, Julian Lewis, Martin Raff, Keith Roberts and James D. Watson, Garland Publishing Inc., 1989, which is herein incorporated by reference. The gene function may be inhibited in any manner known to those of skill in the art, and this inhibition may or may not be directly detectable through routinely used methods. Direct detection of the inhibited function is not necessary, although it may be desirable in some embodiments. If detection of inhibited function is desired, it may be made by any technique or combination of techniques known to those of skill in the art, including quantifiable and non-quantifiable techniques. By way of non-limiting example, inhibition of CCR5 function may be detected through functional assays, binding assays, antibody-based detection methods and/or RNA expression assays. Inhibition of CCR5 function may be detected as a single assay or as part of a larger assay such as an array-based assay that detects function of multiple genes, proteins or RNA transcripts at once. By way of non-limiting example, inhibition may occur at the level of gene transcription, resulting in a reduced level of RNA produced from the gene and therefore inhibited protein function. As a further example, altering DNA may result in an inhibition of function of at least one CCR5 gene that includes a reduction of CCR5 protein expression on the cell surface. Reduced gene transcription of the CCR5 gene has been observed to result from alterations in non-coding regions of the gene, as described in Mummidi et al., “The Human CC Chemokine Receptor 5 (CCR5) Gene”, Journal of Biological Chemistry, 272(49): 30662-30671 (1997) and McDermott et al., “CCR5 Promoter Polymorphism and HIV-1 Disease Progression”, Lancet 352:866-870 (1998) which are herein incorporated by reference. Inhibition of gene function may arise from an alteration that causes a truncation in RNA transcription, such as that described by Quillent et al., “HIV-1-Resistance Phenotype Conferred by Combination of Two Separate Inherited Mutations of CCR5 Gene”, Lancet 351:14-18 (1998), which is herein incorporated by reference. Another example is inhibition that results in the reduction of gene product translation, resulting in the reduced production of protein product from the gene relative to levels of protein production from a non-altered version of the gene. In some embodiments, the inhibition will reduce the protein processing or maturation of the translated protein product, resulting in reduced functional levels of the gene product. For examples of this type of alteration, see Farzan et al., “Tyrosine Sulfation of the Amino Terminus of CCR5 Facilitates HIV Entry”, Cell 96:667-676 (1999), which is herein incorporated by reference. In some embodiments, the inhibition of function of at least one CCR5 gene relative to an unaltered version of the gene results in a reduction of cell surface expression of CCR5 protein molecules. As an example, it has been recognized that at least one deletion within the coding region of CCR5 leads to a protein product which leads to reduced cell surface protein expression as described in Samson et al., “Resistance to HIV-1 Infection in Caucasian Individuals Bearing Mutant Alleles of the CCR-5 Chemokine Receptor Gene”, Nature 382:722-725 (1996) and Benkirane et al., “Mechanism of Transdominant Inhibition of CCR5-Mediated HIV-1 Infection by CCR5A32”, Journal of Biological Chemistry, 272(49): 30603-30606 (1997), which are herein incorporated by reference. It has also been recognized that a DNA alteration that results in a single amino acid substitution in the CCR5 gene can result in reduced protein expression as described in Tamasauskas et al., “A Homologous Naturally Occurring Mutation in Duffy and CCR5 Leading to Reduced Receptor Expression”, Blood 97(11):3651-3654 (2001), which is herein incorporated by reference.

Inhibition of CCR5 gene function has been shown to decrease the potential of cells to be infected with some infectious agents, and with the inhibition of some types of infection. In particular, reduced CCR5 protein function has been shown to reduce the potential of cells to be infected with some types of HIV and therefore to reduce or slow the onset of AIDS symptoms. See Liu et al., “Homozygous Defect in HIV-1 Coreceptor Accounts for Resistance of Some Multiply-Exposed Individuals to HIV-1 Infection”, Cell 86:367-377 (1996); Quillent et al., “HIV-1-Resistance Phenotype Conferred by Combination of Two Separate Inherited Mutations of CCR5 Gene”, Lancet 351:14-18 (1998); Tamasauskas et al., “A Homologous Naturally Occurring Mutation in Duffy and CCR5 Leading to Reduced Receptor Expression”, Blood 97(11):3651-3654 (2001); Samson et al., “Resistance to HIV-1 Infection in Caucasian Individuals Bearing Mutant Alleles of the CCR-5 Chemokine Receptor Gene”, Nature 382:722-725 (1996); Dean et al., “Genetic Restriction of HIV-1 Infection and Progression to AIDS by a Deletion Allele of the CKR5 Structural Gene”, Science 273:1856-1862 (1996); Cormier et al., “Specific Interaction of CCR5 Amino-Terminal Domain Peptides Containing Sulfotyrosines with HIV-1 Envelope Glycoprotein gp120”, Proceedings of the National Academy of Sciences 97(11): 5762-5767 (2000); Kostrikis et al., “A Polymorphism in the Regulatory Region of the CC-Chemokine Receptor 5 Gene Influences Perinatal Transmission of Human Immunodeficiency Virus Type 1 to African-American Infants”, Journal of Virology 73(12): 10264-10271 (1999) and Farzan et al., “Tyrosine Sulfation of the Amino Terminus of CCR5 Facilitates HIV Entry”, Cell 96:667-676 (1999), which are herein incorporated by reference. However, inhibition of CCR5 gene function may also increase the potential of cells to be infected by other types of infectious agents. See Glass et al., “CCR5 Deficiency Increases Risk of Sympomatic West Nile Virus Infection”, Journal of Experimental Medicine 203(1): 35-40 (2006), which is herein incorporated by reference. Therefore, the selection of specific DNA alteration(s) will depend on the particular application in any given embodiment.

A cell is “non-terminally differentiated” when it has the potential to differentiate into a cell type that has less cellular differentiation potential than the parental cell, including but not limited to a terminally differentiated cell. As used in this context, differentiation refers to the potential for a cell to alter over time or to divide so that the resulting cell or cells is expected to have less potential to develop along a plurality of lineages than the original cell. In some situations, the non-terminally differentiated cell is in a lineage with well-defined differentiation potential while in others the differentiation potential is present although its specific nature is unclear or undefined. In some embodiments, the non-terminally differentiated cell is multipotent, totipotent, pluripotent, oligopotent, a stem cell, a spermatocyte, an oocyte, a progenitor cell and/or an established cell line. In some embodiments, the non-terminally differentiated cell is a hematopoetic cell. In some embodiments, stimulus is provided for the differentiation of the altered cell, which may be of any type that is intended to stimulate differentiation of the cell, including differentiation directed toward a well defined cell type or one that is not previously identified or defined. Stimulus provided for the differentiation of the altered cell may be of any type known to those of skill in the art, including chemical stimulus such as retinoic acid, mechanical stimulus such as mechanical force, stretch or contact, or electrical stimulus. In some embodiments providing stimulus for differentiation of the altered cell includes interaction with at least one cell that is more highly differentiated than the altered cell. Interaction includes contact between the cells as well as indirect signaling factors, peptides and other factors. In some embodiments, providing stimulus for differentiation of the altered cell includes administration of at least one cytokine. Depending on the embodiment, a non-limiting group of representative cytokines could include SCF, Flt-3L, IL-3, IL-6, G-CSF, BMP-4, VEGF and/or TGFβ. In some embodiments, the stimulus will be made through non-cellular means such as the addition of a chemical to cell culture media, while in others the stimulus may be introduced via cellular interaction, either between cells or between a cell and a non-cellular presence. In some embodiments, the stimulus will be provided ex-vivo while in others it may be provided in vivo contemporaneously with introduction of the altered cell into a subject. Any method as disclosed herein or as known to those of skill in the art may be used to provide stimulus for differentiation of the altered cell, including those described in U.S. Pat. No. 5,736,396 to Bruder et al. and U.S. Pat. No. 7,045,353 to Benvenisty, which are herein incorporated by reference.

In some embodiments, providing stimulus for differentiation of the altered cell includes providing stimulus for the lineal descendants of the altered cell. As used in this context, “lineal descendants” refers to progeny cells that derive from the altered cell, such as daughter cells. In some embodiments, methods disclosed herein involve maintaining at least one cell ex-vivo in a non-terminally differentiated state. As used in this context, maintaining at least one cell ex-vivo refers to sustaining at least one cell outside of the body of an organism for some quantifiable period of time. This time period may be very brief, on the order of minutes or seconds, or it may be of extended duration. Maintaining at least one cell ex-vivo includes such techniques as sustaining cells obtained during a phlebotomy in a storage unit for a period of a few minutes as well as ongoing cell culture over days, weeks or months. Maintaining at least one cell ex-vivo also includes any period spent, for instance, in propagating a cell culture to confirm the presence of a feature of the cells, confirming the alteration, expansion and/or viable maintenance of a cell during transport. As a non-limiting example, if maintaining at least one cell ex-vivo includes expansion in a given embodiment, techniques such as those described in U.S. Pat. No. 6,436,387 to Bauer et al., as well as those described in U.S. Pat. No. 6,413,509 to Bauer et al. (herein incorporated by reference) may be used during the expansion. In some embodiments, the stimulus provided to the altered cell is designed to encourage differentiation along an identified differentiation pathway. In some embodiments, providing stimulus for the differentiation of the altered cell includes contact with at least one cell that is more highly differentiated than the altered cell. Techniques for maintaining cells in a variety of differentiation states are widely known and practiced. Some examples include those described in U.S. Pat. No. 7,011,828 to Reubinoff et al., U.S. Pat. No. 5,672,346 to Srour et al., and U.S. Pat. No. 6,071,691 to Hoekstra et al., which are herein incorporated by reference.

In some embodiments, providing stimulus for differentiation of the altered cell includes regulation of action of at least one transcription factor. Also included are methods wherein maintaining the cell ex-vivo in a non-terminally differentiated state involves regulation of action of at least one transcription factor. In some embodiments, regulating differentiation of the cell includes regulating at least one transcription factor protein. In some embodiments, regulation of action of transcription factor proteins includes regulating the action of the SOX2 and/or the OCT4 proteins. In some embodiments, the stimulus provided to the altered cell is designed to regulate activity of at least one transcription factor. More information regarding the effects of the SOX2 and OCT4 proteins specifically and transcription factors more generally on cellular differentiation may be found in Kuroda et al., “Octamer and SOX Elements are Required for Transcriptional cis Regulation of Nanog Gene Expression”, Molecular and Cellular Biology 25(6): 2475-2485 (2005) and Boyer et al., “Core Transcriptional Regulatory Circuitry in Human Embryonic Stem Cells”, Cell 122:1-10 (2005), which are herein incorporated by reference. In some embodiments, the non-terminally differentiated cell is derived from a first source and the altered cell is introduced into a subject. The first source may be human. The cell may originate with a human. By way of non-limiting example, the first source may include bone marrow, peripheral blood, epithelial tissue and/or mesenchymal tissue. The cell derived from the first source may be used directly after isolation from the source or it may have undergone some prior processing, such as purification or cell culture. The subject may be human. The subject may be a mammal. The subject may not be the first source in some embodiments. The subject may either be the same entity as the first source or it may be a separate entity. Where the subject and first source are different entities, they may be from the same species or they may be from distinct species. Introducing cells, such as the altered cells, into a subject may be performed according to any manner that is known to those of skill in the art, including transfusion, injection, transdermal applications, nasally or via mucous membranes. As will be apparent to one of skill in the art, introducing the cells may be accompanied by methods to reduce an immune system response, including use of drug therapies and/or irradiation. Cells may also be selected in part due to their expected immunogenicity, including for example cells taken from cord blood, embryonic stem cells, autologous cells and/or cells that have been modified to alter their immunogenicity.

Altered cells may also be introduced into a subject in a manner that encourages or promotes the localization of cells to a specific tissue or region of the body. Cells that are introduced into a subject may also be targeted or directed to a specific site in the body. For example, targeting the cell to a specific site may include administering to the subject at least one agent from a list including a chemokine, a cytokine, a glycoconjugate, a binding protein, an antibody and/or a receptor agonist. Cells may be directed to any site or tissue in the body, including for example the bone marrow, brain or intestine. The altered cell may be introduced after the subject has been treated with myeloablative, immunoablative and/or non-myeloablative conditioning. For example, the subject may be prepared through the use of ionizing radiation to deplete the subject's existing viable cells and therefore reduce competition with the introduced cells.

Also provided are cell lines produced by altering DNA of a non-terminally differentiated cell in a location that inhibits function of at least one CCR5 gene relative to an unaltered version of the gene and providing stimulus for differentiation of the altered cell. Cell lines may also be produced by methods wherein altering DNA results in an inhibition of function of at least one CCR5 gene that includes a reduction of CCR5 protein expression on the cell surface. Cell lines also include those produced by maintaining a cell ex-vivo in a non-terminally differentiated state, altering DNA of the cell in a manner that inhibits function of at least one CCR5 gene relative to an unaltered version of the gene and providing stimulus for differentiation of the altered cell. Cell line production may include those wherein inhibiting function of at least one CCR5 gene relative to an unaltered version of the gene results in a reduction of cell surface expression of CCR5 protein molecules. Further cell lines include those produced by creating a genomic deletion in at least one CCR5 gene in a non-terminally differentiated cell and regulating differentiation of the cell, including cell lines where the non-terminally differentiated cell is a hematopoetic cell.

Also provided are ex-vivo cell lines comprising a plurality of cells containing DNA structure that includes an introduced alteration in at least one CCR5 gene and at least one cell in a differentiated state. In some embodiments, the introduced alteration in at least one CCR5 gene includes at least one of a deletion, a substitution, an inversion, an insertion, hypermethylation, hypomethylation or a combination of these. In some embodiments, the introduced alteration will be detectable using standard molecular biology techniques. In some embodiments, the cell line is derived from a human. The cell line may include cells in a differentiated state that includes at least one induced differentiation state. As is known to those of skill in the art, it is possible to distinguish cells that have been induced to differentiate by a variety of means. Any specific detection means used to distinguish cells that are in an induced differentiation state would depend on the specific situation and goals of the user. Methods to distinguish cells that have been induced to differentiate include the use of microarrays, 2-dimensional gel electrophoresis, the presence or absence of specific proteins either on the cell surface or in secreted form, and the decreased capacity to respond to other cellular stimulus. As an example of the use of microarrays to distinguish cells that have been induced to differentiate from those that have not been induced to differentiate, see Bhattacharya et al. “Comparison of the gene expression profile of undifferentiated human embryonic stem cell lines and differentiating embryoid bodies”, BMC Developmental Biology 5:22 (2005), which is herein incorporated by reference. As an example of the use of 2-dimensional gel electrophoresis to distinguish cell lines that have been induced to differentiate with different PPAR-agonist drugs, see Bottoni et al., “A two-dimensional electrophoresis preliminary approach to human hepatocarcinoma differentiation induced by PPAR-agonists”, J. Cell Mol. Med., 9(2):462-467 (2005). As an example of the detection of the presence or absence of specific proteins expressed by cell lines induced to differentiate with different classes of drugs, see Lyakh et al., “TGF-β and vitamin D₃ utilize distinct pathways to suppress IL-12 production and modulate rapid differentiation of human monocytes into CD83+dendritic cells”, J. Immunology 174:2061-2070 (2005).

Further illustration of the methods disclosed herein may be found in the Figures, as described below.

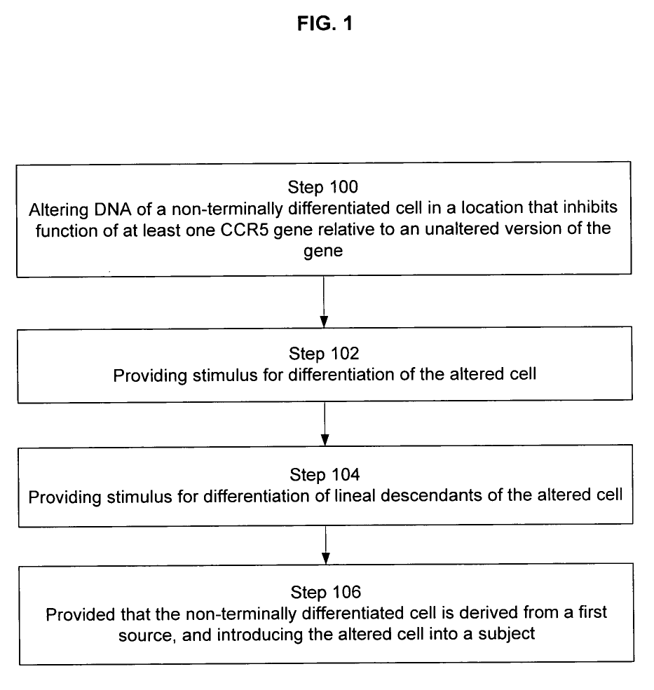

FIG. 1 presents an illustrative method. First step 100 includes altering DNA of a non-terminally differentiated cell in a location that inhibits function of at least one CCR5 gene relative to an unaltered version of the gene. In one approach, altering DNA of a non-terminally differentiated cell in a location that inhibits function of at least one CCR5 gene relative to an unaltered version of the gene may include creating a genomic deletion in at least one CCR5 gene in a non-terminally differentiated cell. Altering DNA of a non-terminally differentiated cell in a location that inhibits function of at least one CCR5 gene relative to an unaltered version of the gene may further include regulating differentiation of the cell subjected to genomic deletion. At a second step 102 the method includes providing stimulus for differentiation of the altered cell. Step 104 includes providing stimulus for differentiation of the lineal descendants of the altered cell. The stimulus provided may be any known to those of skill in the art or as described herein. As used herein, “lineal descendants” includes descendant cells in a more advanced state of differentiation as the altered cell of step 102 as well as those not in a more advanced state of differentiation. Step 106 provides that the non-terminally differentiated cell is derived from a first source, and introducing the altered cell into a subject. The first source may be a cell culture or mammalian tissue. The subject may be the same source as the first source, or it may be a different entity. Depending on the embodiment, the stimulus provided as described in steps 102 and 104 may occur before or after the introduction into a subject.

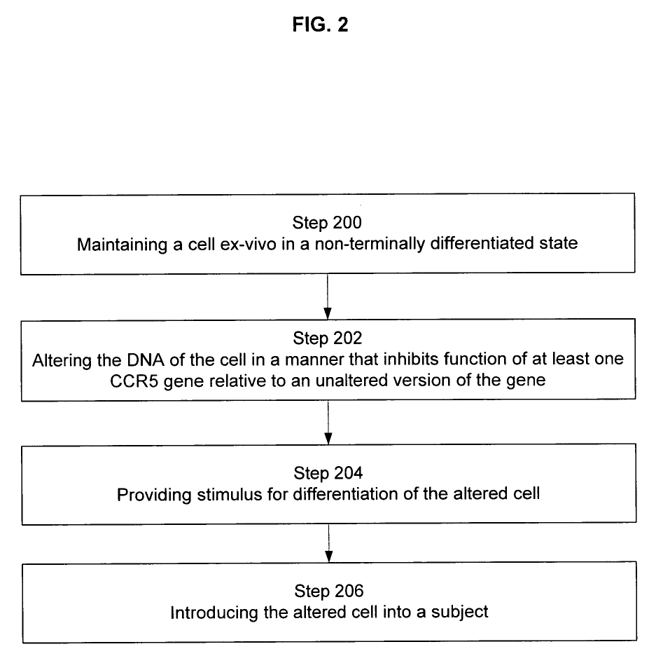

In another illustrative example shown in FIG. 2, a method as described herein includes maintaining a cell ex-vivo in a non-terminally differentiated state at step 200. This cell may have been maintained ex-vivo in a non-terminally differentiated state for several passages in cell culture or it may have been recently obtained. The non-terminally differentiated cell may be a hematopoetic cell. The method also includes at step 202 altering the DNA of the cell in a manner that inhibits function of at least one CCR5 gene relative to an unaltered version of the gene. This may be carried out in any manner as described herein or as known to those of skill in the art. At step 204, the method includes providing stimulus for differentiation of the altered cell. This stimulus may include regulation of action of at least one transcription factor, or be designed to regulate the activity of at least one transcription factor. The stimulus may be designed to encourage differentiation along an identified differentiation pathway. The stimulus may include contact with at least one cell that is more highly differentiated than the altered cell. At step 206 the method includes introducing the altered cell into a subject. Although the steps shown in FIG. 2 are represented in a linear order, they need not be carried out in that order. For example, step 206 may be carried out before step 204.

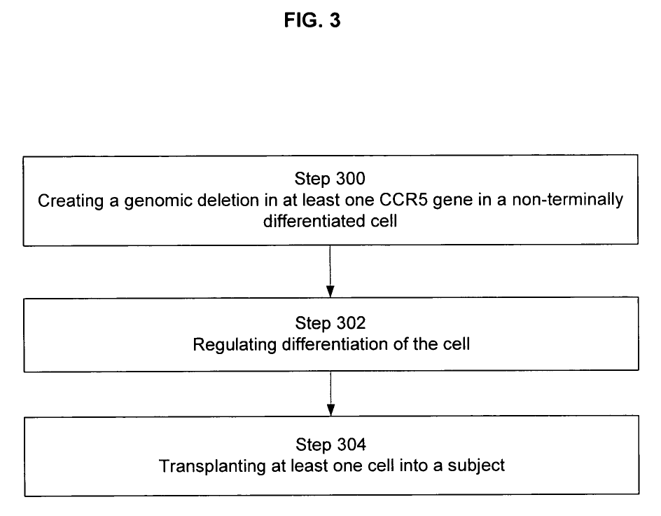

As shown in FIG. 3, methods as described herein include creating a genomic deletion in at least one CCR5 gene in a non-terminally differentiated cell as described in step 300. This deletion may be created by any means as described herein or known to those of skill in the art. Step 302 includes regulating differentiation of the cell. This regulation may include providing stimulus designed to encourage differentiation of the cell, providing stimulus designed to inhibit differentiation of the cell or otherwise alter the differentiation of the cell from that expected without additional regulation. Step 304 provides for introducing at least one cell into a subject. This cell may be purified or it may be introduced as part of an unpurified mixture of cells and cellular components.

Further illustration of the methods described herein may be found in the Examples.

### EXAMPLE 1

Methods as described herein are applicable to treatments to reduce HIV infection and/or progression in exposed individuals.

In one approach, a non-terminally differentiated hematopoetic cell may be isolated from an individual. The isolation could be from the peripheral blood or from a tissue such as the bone marrow, and could be easily carried out using routine phlebotomy methods and supplies. After isolation, the cell is maintained in culture for the period necessary to introduce an alteration in at least one CCR5 gene using zinc-finger proteins and double strand breaks to introduce specific alterations in a targeted manner, such as techniques described in Urnov et al., “Highly Efficient Endogenous Human Gene Correction Using Designed Zinc-finger Nucleases” Nature 435: 646-651 (2005). This alteration could be of any type that would lead to the reduction of CCR5 protein expression on the cell surface, as described herein. Once the alteration is introduced into the cell, stimulus for differentiation could be provided. For example, stimulus could be added to the altered cell while it is maintained in cell culture. The cell may also be introduced into a subject and the stimulus introduced either simultaneously with or subsequent to this introduction. The altered cell may be injected into the subject along with factors designed to stimulate the cell along a differentiation pathway or pathways that lead to the expression of CD4 on the cell surface. Alternatively, the cell could be introduced into the subject in such a manner as to provide stimulus for the cell from factors inherently present in, for example, the bone marrow. After introduction into the subject, the cell may further differentiate and the resulting daughter cells would maintain the alteration introduced into the parental cell. As a result, the subject would over time accumulate some number of differentiated cells that express CD4 on the cell surface containing the CCR5 alteration and therefore it would be expected that HIV infection would be less likely to initiate or progress in the subject.

### EXAMPLE 2

Methods as described herein are also applicable as research tools and treatment options generally for pathogens that rely on CCR5 to infect or increase infection in cells. One example of such a pathogen is West Nile Virus (WNV).

The WNV is pathogen that causes serious encephalitis in several species, including humans. For more information regarding the incidence and transmission of WNV in the Americas, see “The West Nile Virus Fact Sheet” from the Pan American Health Organization, Program on Communicable Diseases, September 2002. Human and mouse cells with inhibited function of at least one CCR5 gene have been shown to be more susceptible to infection with WNV than cells without inhibited function. See Glass et al., “CCR5 Deficiency Increases Risk of Symptomatic West Nile Virus Infection”, Journal of Experimental Medicine 203(1): 35-40 (2006).

It is possible to use methods as described herein to create cell lines that would be useful for research into WNV infection, disease progression and transmission. The DNA of a non-terminally differentiated cell from an established cell culture may be altered in a location that inhibits function of at least one CCR5 gene relative to an unaltered version of the gene. The cell culture would be of a type of cell that is susceptible to WNV infection, such as brain cells. After the alteration, the culture may be maintained so that a group of daughter cells of the same cell lineage containing similar alterations in their CCR5 genes would be established as a single or as a set of cell cultures. The group(s) of cells may then be maintained in culture over an extended period of time. During culture, stimulus may be provided for differentiation of the cultured cell and/or daughter cell(s). The resulting differentiated cells would be expected to be more susceptible to WNV infection than cells from the same source that are similarly maintained in culture and stimulated to induce differentiation but lack the CCR5 alteration.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All of the above U.S. patents, U.S. patent application publications, U.S. patent applications, foreign patents, foreign patent applications and non-patent publications referred to in this specification and/or listed in any Application Data Sheet, are incorporated herein by reference, in their entireties.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method comprising:
altering DNA of a non-terminally differentiated cell in a location that inhibits function of at least one CCR5 gene relative to an unaltered version of the gene; and
providing stimulus for differentiation of the altered cell.

2. The method as in claim 1 further including:
identifying an alteration corresponding to an inhibition of function of at least one CCR5 gene that includes reduction of CCR5 protein expression on the cell surface,
wherein altering DNA of a non-terminally differentiated cell includes altering DNA according to the identified alteration corresponding to an inhibition of function of at least one CCR5 gene that includes a reduction of CCR5 protein expression on the cell surface.

3. The method as in claim 1 wherein the altering DNA comprises creating at least one of the following: an insertion, a deletion, a substitution, an inversion, a change in methylation state or substantially any combination of these.

4. The method as in claim 1 wherein the altering DNA comprises altering a coding region.

5. The method as in claim 1 wherein the altering DNA comprises altering a non-coding region.

6. The method as in claim 1 wherein the altering DNA comprises altering a DNA sequence within at least one CCR5 gene with a 32 nucleotide deletion encompassing nucleotides 794 through 825 of the sequence with GenBank accession number NM_{—}000579.

7. The method as in claim 1 wherein the altering DNA creates a DNA sequence within at least one CCR5 gene that encodes a protein sequence with a frameshift after codon 174 and a truncation at codon 206 relative to the sequence of NCBI Entrez Protein database accession number P51681.

8. The method as in claim 1 wherein the providing stimulus for differentiation of the altered cell includes regulation of action of at least one transcription factor.

9. The method as in claim 1 further comprising:
providing stimulus for differentiation of lineal descendants of the altered cell.

10. The method as in claim 1 further comprising:
providing that a non-terminally differentiated cell is derived from a first source; and
introducing the altered cell into a subject.

11. The method as in claim 10 wherein the subject is not the first source.

12. The method as in claim 10 wherein the subject is human.

13. The method as in claim 10 wherein the first source is human.

14. An altered cell line produced by the method of claim 1.

15. A method comprising:
maintaining a cell ex-vivo in a non-terminally differentiated state;
altering DNA of the cell in a manner that inhibits function of at least one CCR5 gene relative to an unaltered version of the gene; and
providing stimulus for differentiation of the altered cell.

16. The method as in claim 15 wherein the altering DNA is altering genomic DNA.

17. The method as in claim 16 wherein the altering genomic DNA comprises at least one of the following: creating a deletion, creating a substitution, creating an insertion, creating an inversion, a change in methylation state or substantially any combination of these.

18. The method as in claim 15 wherein the altering DNA occurs ex-vivo.

19. The method as in claim 15 wherein the inhibits function of at least one CCR5 gene relative to an unaltered version of the gene results in a reduction of cell surface expression of CCR5 protein molecules.

20. The method as in claim 15 wherein the cell originates from a human.

21. The method as in claim 15 further comprising:
introducing the altered cell into a subject.

22. The method as in claim 21 wherein the subject is a human.

23. The method as in claim 21 wherein the cell does not originally derive from the subject.

24. The method as in claim 15 wherein the maintaining the cell ex-vivo in a non-terminally differentiated state involves regulation of action of at least one transcription factor.

25. The method as in claim 15 wherein the providing stimulus for the differentiation of the altered cell includes administration of at least one cytokine.

26. The method as in claim 15 wherein the stimulus provided to the altered cell is designed to encourage differentiation along an identified differentiation pathway.

27. The method as in claim 15 wherein the stimulus provided to the altered cell is designed to regulate activity of at least one transcription factor.

28. The method as in claim 15 wherein the providing stimulus for the differentiation of the altered cell includes interaction with at least one cell that is more highly differentiated than the altered cell.

29. An altered cell line produced by the method of claim 15.

30. A method comprising:
creating a genomic deletion in at least one CCR5 gene in a non-terminally differentiated cell; and
regulating differentiation of the cell.

31. The method as in claim 30 wherein the non-terminally differentiated cell is a hematopoetic cell.

32. The method as in claim 30 wherein the regulating differentiation of the cell includes regulating at least one transcription factor protein.

33. The method as in claim 32 wherein the regulating at least one transcription factor protein includes regulating the action of the SOX2 protein.

34. The method as in claim 32 wherein the regulating at least one transcription factor protein includes regulating the action of the OCT4 protein.

35. The method as in claim 30 wherein the regulating differentiation of the cell includes administration of at least one cytokine.

36. The method as in claim 30 further comprising: introducing at least one cell into a subject.

37. The method as in claim 36 wherein the subject is a mammal.

38. The method as in claim 36 wherein the subject is a human.

39. An ex-vivo cell line comprising:
a plurality of cells containing DNA structure that includes an introduced alteration in at least one CCR5 gene; and
at least one cell in a differentiated state.

40. A cell line as in claim 39 wherein the differentiated state includes at least one induced differentiation state.

41. The cell line as in claim 39 wherein the cell line is derived from a human.

42. The cell line as in claim 39 wherein the introduced alteration in at least one CCR5 gene includes at least one of a deletion, a substitution, an inversion, an insertion, hypermethylation, hypomethylation or a combination of these.
